(19) United States
(12) Patent Application Publication
(10) Pub. No.: US 2016/0046557 A1
(43) Pub. Date: Feb. 18, 2016

(54) PROCESS FOR THE PREPARATION OF VINYL ACETATE

(71) Applicant: Wacker Chemie AG, Munich (DE)

(72) Inventors: Mehmet GUENALTAY, Emmerting (DE); Peter SCHULTHEISS, Burghausen (DE); Heribert WESTERMAYER, Burghausen (DE)

(73) Assignee: Wacker Chemie AG, Munich (DE)

(21) Appl. No.: 14/780,364

(22) PCT Filed: Mar. 26, 2014
(86) PCT No.: PCT/EP2014/056067

§ 371 Date: Sep. 25, 2015

(30) Foreign Application Priority Data
Mar. 27, 2013 (DE) 10 2013 205 492.0

(51) Int. Cl. C07C 67/54; C07C 67/05

(52) U.S. Cl. CPC C07C 67/54; C07C 67/05

(57) ABSTRACT

A process for the preparation of vinyl acetate by a heterogeneously catalysed, continuous gas-phase reaction of ethylene, acetic acid and oxygen in a reactor, where process heat liberated during the reaction is removed from the reactor by means of heat exchange with water, generating intrinsic steam, the product mixture leaving the reactor and comprising ethylene, vinyl acetate, acetic acid, water, carbon dioxide and inert gases is separated by distillation using one or more azeotrope columns and/or one or more pure distillation columns, wherein at least one azeotrope column and/or pure distillation column contains packings, and intrinsic steam is used at least partially for introducing energy into the thus-equipped azeotrope columns and/or pure distillation columns.

### CROSS REFERENCE TO RELATED APPLICATIONS

This application is the U.S. National Phase of PCT Appln. No. PCT/EP2014/056067 filed Mar. 26, 2014, which claims priority to German Application No. 10 2013 205 492.0 filed Mar. 27, 2013, the disclosures of which are incorporated in their entirety by reference herein.

### BACKGROUND OF THE INVENTION

1. Field of the Invention

The invention relates to processes for the preparation of vinyl acetate in a heterogeneously catalysed, continuous gas phase process by reacting ethylene with acetic acid and oxygen while utilizing the process heat liberated during the process.

2. Description of the Related Art

The preparation of vinyl acetate by reacting ethylene with acetic acid and oxygen or oxygen-containing gases over a fixed-bed catalyst in the gas phase has been known for a long time and is described for example in Ullmann's Encyclopedia of Industrial Chemistry. The starting materials are converted to vinyl acetate in an exothermic reaction generally at a pressure from 5 to 15 bar and a temperature from 120° C. to 200° C. in a fixed-bed tubular reactor or fluidized-bed reactor corresponding to the following empirical formula:

C₂H₄+CH₃COOH+½O₂−>CH₃COOCH═CH₂+H₂O

The product mixture emerging from the reactor contains, besides vinyl acetate, essentially unreacted starting materials and water, as well as inert materials and byproducts such as carbon dioxide, acetaldehyde, methyl acetate and ethyl acetate. Inert materials are essentially nitrogen, argon, methane and ethane and are introduced into the process as impurities in the starting materials. Water is also formed from the secondary reaction of ethylene and oxygen to give carbon dioxide. Separating off the water and the ethyl acetate in particular requires a high energy expenditure.

After leaving the reactor, reaction products from the product mixture and unreacted acetic acid from the circulating gas are condensed out and passed to the work-up. The remaining gas is purified by carbon dioxide, then admixed with starting materials, brought to reaction temperature by means of heat exchangers driven by heating steam and returned to the reactor. The condensed-out products vinyl acetate and water, as well as unreacted acetic acid and byproducts are separated from one another in a multistage, usually steam heated, distillation process. Customary distillation steps are, for example, dewatering, azeotropic distillation, pure distillation, byproduct removal, waste water purification, residue processing or separation of low-boiling components and high-boiling components. A very wide range of variants are known for the distillation process.

The reaction conditions in the reactor are regulated by means of evaporative water cooling, to temperatures from 120° C. to 200° C. and pressures from 5 to 15 bar. The water of the evaporative cooling is converted to steam, so-called “intrinsic steam,” which usually has a temperature of 120° C. to 170° C. at a pressure from 2 to 8 bar. Fractions of the intrinsic steam are often used for heating individual process steps of the vinyl acetate preparation, such as for heating individual distillation columns for separating the product mixture.

One problem here is that the intrinsic steam obtained in this way has a relatively low temperature and pressure level and consequently, without heating or compressing, can only be used for heating some of the process steps of the known processes for vinyl acetate preparation, such as heat treatment of the dewatering column, the ethyl acetate column or the low-boiling component column, or else waste water purification, residue processing, the circulating gas heater, or the acetic acid evaporator or heater. However, the entire amount of the resulting intrinsic steam is not required for operating these plant components, and so some of the intrinsic steam remains unused. For the other process steps, such as the azeotropic distillation or the pure distillation, a heating steam was hitherto necessary which usually has a temperature of up to 250° C. and therefore has a higher temperature level than the intrinsic steam made available from known vinyl acetate preparation processes, and consequently had to be supplied from external sources. Overall, only up to 80% by weight of the intrinsic steam from the vinyl acetate preparation could hitherto be utilized for the purpose of heating in the same process.

The fraction of the intrinsic steam that cannot be utilized in the vinyl acetate preparation has hitherto mostly been condensed, which leads to complete energy loss, or supplied to other operations within the framework of a production network. However, this is associated with organizational and apparatus expenditure or is in many cases not possible at all due to lack of need. The use of intrinsic steam for heating product pipelines or buildings is subject to seasonal variations and is consequently also not suited to a permanently complete utilization of intrinsic steam.

To completely utilize intrinsic steam in the vinyl acetate preparation process, DE-A 102005054411 recommends compressing the intrinsic steam by means of steam jet-steam compressors using external high-pressure steam. Often, considerable amounts of valuable high-pressure steam are required for this purpose, meaning that more steam is generated in this way than can ultimately be utilized in the vinyl acetate preparation process and consequently there continues to be a provision problem for steam. Moreover, compression is associated with technical expenditure and renders process control more complex also when running up the plant.

### SUMMARY OF THE INVENTION

Against this background, the object was to provide processes for the preparation of vinyl acetate in which the intrinsic steam formed in these processes from the removal of the reaction energy can be utilized as completely as possible in the same vinyl acetate preparation process.

Surprisingly, the object was achieved through the use of at least one azeotrope column and/or at least one pure distillation column containing packings, and at least some of the intrinsic steam produced in the vinyl acetate preparation process is used for introducing energy into a thus-equipped azeotrope column and/or pure distillation column. It was particularly surprising here that despite these measures the desired separation efficiency was achieved and any impurities arose to no noteworthy degree or only to the customary degree.

### DETAILED DESCRIPTION OF THE PREFERRED EMBODIMENTS

The invention thus relates to processes for the preparation of vinyl acetate by means of heterogeneously catalysed, continuous gas-phase reaction of ethylene, acetic acid and oxygen in a reactor, where process heat liberated during the reaction is removed from the reactor by means of heat exchange over water and as a result intrinsic steam is formed, and separating the product mixture leaving the reactor and comprising ethylene, vinyl acetate, acetic acid, water, carbon dioxide and inert gases, by means of distillation using one or more azeotrope columns and/or one or more pure distillation columns, characterized in that at least one azeotrope column and/or at least one pure distillation column contains packings and the intrinsic steam is used at least partially for introducing energy into one or more thus-equipped azeotrope columns and/or pure distillation columns.

The continuous preparation of vinyl acetate is preferably performed in tubular reactors which are charged with a fixed-bed catalyst, or in fluidized-bed reactors with catalysts suitable for the fluidized bed. The catalysts used are generally supported catalysts doped with precious metals or precious metal salts and promotors, for example bentonite beads doped with palladium chloride and with gold, cadmium and potassium salts. The reactor is charged with ethylene, oxygen and acetic acid and the reaction is preferably carried out at a pressure from 5 to 15 bar, preferably 5 to 10 bar, and a temperature of preferably 120° C. to 200° C., in particular 150° C. to 180° C.

The temperature in the reactor can be adjusted in the customary manner by evaporative water cooling. Here, as is known, water evaporates, and so-called intrinsic steam (IS) is formed. This can remove the process heat, i.e. the heat liberated in the course of the chemical reactions in the reactor, also called reaction energy, from the reactor.

Usually, 1.1 to 1.6 and in particular 1.2 to 1.5 tonnes of intrinsic steam are formed per tonne of vinyl acetate formed. The intrinsic steam preferably has a temperature of 120° C. to 170° C. and more preferably 140° C. to 160° C. The pressure of the intrinsic steam is preferably 2 to 8 bar and more preferably 3.5 to 5.5 bar. Consequently, the intrinsic steam has the pressure level of so-called medium-pressure steam and thus a lower pressure level than customary high-pressure steam and a higher pressure level than customary low-pressure steam.

To clarify, it may be noted that all pressure data in the present application relate to absolute pressure. Absolute pressure data refer to the pressure relative to the perfect vacuum, i.e. compared to the absolutely molecule-free space.

Relative pressure data, by contrast, indicate the pressure difference relative to the respective ambient pressure.

Preferably, 10 to 60% by weight, more preferably 15 to 30% by weight, of the intrinsic steam formed overall is used for introducing energy into the azeotrope column and/or the pure distillation column. Preferably, 0.3 to 0.6 and more preferably 0.4 to 0.5 tonnes of intrinsic steam are used per tonne of vinyl acetate formed for introducing energy into the azeotrope column and/or the pure distillation column. The remaining fraction of the intrinsic steam is generally utilized for plant components whose energy introduction is serviced with low-pressure steam or medium-pressure steam, such as in particular the dewatering column, the ethyl acetate column, the low-boiling component column, waste water purification, residue processing, the circulating gas heater or the acetic acid evaporator or heater.

Preferably, the total amount of the intrinsic steam liberated in the process for the preparation of vinyl acetate is thus utilized in the same process.

The amount of process heat also depends on the ethylene selectivity. The ethylene selectivity characterizes the selectivity of the reaction of ethylene to vinyl acetate in the reactor at the particular point while carrying out the process and is calculated as molar ratio of the vinyl acetate formed in the reactor at the particular point, based on vinyl acetate and carbon dioxide. The ethylene selectivity depends on the process conditions and in particular also on the activity of the catalyst and is consequently generally not constant while carrying out the process of vinyl acetate preparation. In general, the ethylene selectivity is 91 to 96%. The specific process heat that forms in the reactor is accordingly usually 0.907 to 0.660 Mwh per tonne of vinyl acetate formed.

It is essential to the invention that at least one azeotrope column and/or at least one pure distillation column contains packings.

Packings which can be used are the packings customary in chemical process technology. Thus, the packings are for example based on metallic or ceramic materials or on plastic. Preference is given to metallic materials. Examples of metallic materials are iron, such as steel, in particular stainless steel, copper, brass, aluminium, nickel, Monel metals or titanium. Examples of ceramic materials are oxides of the main group metals or semi-metals, for example of boron, aluminium or silicon, in particular borosilicate glasses or aluminosilicate glasses. Examples of plastics are polyolefins, halogen-substituted polyolefins, polyethersulphones, polyphenylene sulphides or polyaryl ether ketones. The packings can be in a very wide variety of forms, such as hollow-cylinders, also called rings, or in saddle or bead form. Preference is given to Pall rings, Berl saddles, Hiflow rings, Intalox saddles, Igel or in particular Raschig super rings. The packings have one or more diameters from preferably 5 to 100 mm, more preferably 10 to 80 mm and most preferably 25 to 50 mm. The packings preferably have a specific surface area from 80 to 350 m²/m³ and more preferably from 100 to 250 m²/m³. The specific surface area is calculated here by multiplying the surface area of the material from which a packing is formed, by the number of these packings, based on one cubic meter of the bed.

The packings can be used as an ordered packing or preferably as a dumped bed. In a dumped bed, as is known, a large number of packings lie in loose and disordered layering on support elements, such as perforated support grids or other support trays or grids. When using a plurality of support elements, a plurality of layers of packings can be installed (beds). Ordered packings have a regularly shaped structure, such as, for example, fabric or sheet metal ordered packings, in particular thin, corrugated or perforated plates or meshes, for example made of metal, plastic, glass or ceramic. The respective process gas flows as usual through the dumped bed or ordered packing. The ordered packings have a specific surface area from preferably 100 to 750 m²/m³ and more preferably from 150 to 350 m²/m³. The specific surface area is calculated here from the surface area of the material from which the ordered packing is shaped, based on one cubic metre of the ordered packing.

Packings are preferably located in the rectification section of the azeotrope column. Packings are preferably located in a region above the feed point. The feed point is the point in the azeotrope column at which the mixture to be separated is introduced into the azeotrope column. The packings are preferably located directly above the feed point or above a tenth tray, counting from the feed point of the azeotrope column. The packings are preferably located above the uppermost tray of the azeotrope column. Here, the uppermost tray is the tray which is closest to the top of the azeotrope column. The total number of trays in the azeotrope column is preferably 30 to 80, more preferably 30 to 70 and most preferably 40 to 60. The trays are preferably attached between the bottom of the azeotrope column up to the point of the azeotrope column at which, when viewed from the bottom, the first packings are incorporated. The trays are thus preferably located below the packings. No trays are thus preferably attached above the packings.

The trays customary in chemical process technology, such as, for example, bubble trays, valve trays, sieve trays, tunnel trays or fixed-valve trays, can be used.

The pressure at the top of the azeotrope column (head pressure) is preferably more than 600 and up to 1500 mbar, more preferably 800 to 1000 mbar, yet more preferably 800 to 950 mbar and most preferably 850 to 935 mbar. The pressure at the bottom of the azeotrope column (sump pressure) is preferably 650 to 2500 mbar, more preferably 1000 to 2000 mbar, yet more preferably 1000 to 1200 mbar, and most preferably 1050 to 1150 mbar. The difference between the sump pressure and the head pressure is preferably 50 to 1000 mbar, more preferably 100 to 300 mbar, and most preferably 125 to 250 mbar.

The temperature at the bottom of the azeotrope column (sump temperature) is preferably 100 to 130° C. and more preferably 110 to 125° C.

The temperature difference between the intrinsic steam and the gas stream or the liquid to which heat from the intrinsic steam is transferred, is preferably 5 to 25° C., more preferably 10 to 25° C., even more preferably 15 to 25° C., and most preferably 15 to 20° C.

The transfer of energy, in particular heat, from the intrinsic steam into the azeotrope column can take place in different ways, for example using one or more heat exchangers. Customary heat exchangers can be used, such as for example plate heat exchangers, tube (bundle) heat exchangers, U-tube heat exchangers, jacket tube heat exchangers, heating elements or countercurrent bed heat exchangers. Preference is given to tube (bundle) heat exchangers.

Preferably, a liquid is removed from the sump of the azeotrope column, to which liquid heat is then transferred by means of one or more heat exchangers. The liquid heated in this way, optionally in the form of a liquid/steam mixture, is preferably returned to the sump of the azeotrope column. The steam used can be, for example, partially external steam, in particular medium-pressure steam or high-pressure steam, and partially intrinsic steam. The liquid removed is heated herein preferably to a temperature from 110 to 140° C. and more preferably 130 to 140° C.

In an alternative procedure, one or more, preferably one, intermediate evaporators can also be connected to the azeotrope column. Customary intermediate evaporators can be used. Intermediate evaporators are preferably operated with intrinsic steam and optionally additional external steam, more preferably exclusively with intrinsic steam.

Intermediate evaporators are preferably connected to the stripping section of the azeotrope column, i.e. preferably below the feed, and most preferably below the take-off of ethyl acetate. It is most preferred to connect intermediate evaporators between trays 5 and 20, more preferably between trays 7 and 15 and most preferably between trays 8 and 12, counting in each case from the bottom of the azeotrope column. At one or more of the aforementioned positions, liquid is removed from the azeotrope column and supplied to intermediate evaporators. In intermediate evaporators, the liquid removed from the azeotrope column is heated by means of one or more heat exchangers. The liquid heated in this way, optionally in the form of a liquid/steam mixture, is then returned again to the azeotrope column, preferably to the same position or the same zone at which the liquid was removed from the azeotrope column. The operating temperature of the intermediate evaporator is preferably 5to 15° C., more preferably 7 to 10° C., lower than the bottom temperature.

It is also possible for one or more intermediate evaporators and additionally one or more heat exchangers to be connected to the azeotrope column. In such a procedure, the heat exchangers can for example be operated partially with intrinsic steam and partially, preferably exclusively, with external steam, in particular medium-pressure steam or high-pressure steam. Here, heat exchangers are preferably connected to the bottom of the azeotrope column. The bottom of the azeotrope column is heated by means of heat exchangers, preferably to a temperature from 110 to 150° C., more preferably 125 to 140° C. and most preferably 130 to 135° C.

The use of intermediate evaporators makes it possible to operate the azeotrope column at higher pressures within the aforementioned pressure ranges of the azeotrope column. As a result, the energy efficiency of the process according to the invention can be increased.

In the azeotrope column, a mixture comprising essentially acetic acid, ethyl acetate, water and vinyl acetate is usually distilled. Vinyl acetate and water are generally distilled off as an azeotrope and removed at the head or in the region of the head of the azeotrope column.

The feed to the azeotrope column preferably comprises 25 to 50% by weight of vinyl acetate, 5 to 15% by weight of water, 0 to 1% by weight of ethyl acetate and 40 to 70% by weight of acetic acid. In the bottom of the azeotrope column there is preferably a mixture comprising water and acetic acid with a fraction of acetic acid of preferably 90 to 99% by weight. At the top of the azeotrope column, a mixture is preferably removed which comprises 90 to 95% by weight of vinyl acetate and 5 to 10% by weight, in particular 5 to 8% by weight, of water. Ethyl acetate is preferably removed as a side take-off. The side take-off is preferably located in the stripping section of the azeotrope column. The side take-off preferably comprises 0 to 1% by weight of ethyl acetate, more preferably 0.1 to 0.3% by weight of ethyl acetate. The data in % by weight refer to the total weight of the respective mixture or of the feed or of the side take-off.

The azeotrope column can be inserted at different positions of the present process, as described below by way of example.

The product mixture emerging from the reactor essentially comprises vinyl acetate, ethylene, acetic acid, water, oxygen, carbon dioxide, and also the inert materials, such as nitrogen, argon, methane or ethane, and byproducts, such as carbon dioxide, acetaldehyde, methyl acetate and ethyl acetate.

Usually, acetic acid and further condensable reaction products are condensed out of the product mixture (condensate). The remaining gas phase is optionally washed in a scrubber stage with acetic acid (circulatory gas scrubbing). The gas phase washed in this way can be returned to the reactor and the washing solution obtained in the process can be introduced for example into an azeotrope column. Vinyl acetate can be separated from acetic acid, water and other impurities from the condensate and optionally from the washing solution of the circulatory gas scrubbing by means of heating-steam-operated distillation steps. Here, the distillation plants mostly comprise a plurality of columns, such as, for example, a dewatering column, an azeotrope column, a predewatering column or a pure vinyl acetate column. Individual columns can be arranged in different process variants in a different order, as explained below.

In one process variant, the condensate obtained from the product mixture can, for example, be added directly to an azeotrope column. The top steam of the azeotrope column can be cooled and subjected to a phase separation, in which case a water phase and an organic phase are formed. The water phase can be stripped off. The organic phase containing vinyl acetate can optionally be returned in part to the top of the azeotrope column, and be added in its entirety or partially to a dewatering column or be collected as an intermediate in a receiver. The liquid from the receiver can then be further purified in a dewatering column or in a further column. The bottom of the dewatering column can then be transferred to a pure vinyl acetate column, from which pure vinyl acetate is obtained overhead.

In another process variant, the product mixture is fed directly to a predewatering column, from which water, vinyl acetate and optionally further condensable fractions are distilled off overhead and then condensed. Following phase separation of the condensate, the organic phase containing vinyl acetate thus obtained is generally returned in its entirety or in part to the predewatering column, or is added in part to an azeotrope column or to a dewatering column, and then optionally further purified via a pure vinyl acetate column.

It is also possible to dispense with a pure vinyl acetate column. Thus, the product mixture can be fed from the reactor to a predewatering column. The bottom product of the predewatering column can be introduced into the azeotrope column. Water and vinyl acetate and other volatile constituents can be distilled off overhead from the predewatering column, and some of these can be removed following condensation and phase separation into an organic phase and a water phase. The organic phase containing vinyl acetate thus obtained can be returned in its entirety or in part to the predewatering column or be introduced in part into an azeotrope column or dewatering column. The noncondensed constituents of the top product of the predewatering column are placed into a washing column operated with acetic acid (circulatory gas scrubber) and washed. The bottom product of the circulatory gas scrubber is also fed to the aforementioned azeotrope column. An organic phase is formed from the top steam of the azeotrope column following condensation and phase separation, and said phase is finally also added, in its entirety or in part, to the aforementioned dewatering column. Pure vinyl acetate can be obtained from the dewatering column as a side take-off. Alternatively, the bottom product of the dewatering column can be further purified in a pure vinyl acetate column. Finally, pure vinyl acetate can also be obtained as top product of the pure vinyl acetate column.

Using the procedure according to the invention, it becomes possible to utilize the intrinsic steam obtained from the process heat of the vinyl acetate preparation to a greater extent and even in its entirety in the same process. As a result, on the one hand, an intrinsic steam excess and its supply to processes elsewhere or its degradation can be reduced or even avoided entirely. On the other hand, the importing of additional heating steam into the vinyl acetate preparation process can be minimized accordingly, thus reducing the need for foreign heating media. Overall, the operating costs can thus be reduced by the procedure according to the invention.

In this connection, it was particularly surprising that the use of packings in the azeotrope column and/or pure vinyl acetate column in no way lastingly impaired the vinyl acetate preparation, even in the case of a long-term operation of the plant. This is because, for example in the azeotrope column, generally a mixture comprising vinyl acetate, water, acetic acid and ethyl acetate is distilled which, especially in the rectification section of the azeotrope column, has a tendency toward phase separation. Phase separation in a distillation plant counteracts a material separation by means of distillation, in the present case in particular the removal of ethyl acetate. In order to avoid this, the azeotrope column in the prior art was equipped exclusively with separation trays. Separation trays are also less susceptible to contamination which can arise during a long-term operation of a vinyl acetate production plant, and which accumulate over time. In the event of the inventive use of packings in the azeotrope column, none of these problems arose to other than a negligible extent. Moreover, the required separation efficiency was achieved.

The examples below serve to further illustrate the invention without limiting the invention in any way:

The reaction of ethylene with acetic acid and oxygen in the reactor was carried out in the conventional way. The reactor was operated at an inlet pressure of 10.8 bar-abs and a reactor temperature from 160 to 175° C. The selectivity was 92%, the intrinsic steam temperature 145° C., the intrinsic steam pressure 4 bar and the specific intrinsic steam generation 1.45 tonnes of intrinsic steam per tonne of vinyl acetate formed.

### Comparative Example 1

The product mixture was worked up as described in the example of DE-A1 3422575. The azeotrope column accordingly contained no packings. Here, the azeotropic distillation was operated under atmospheric pressure; the bottom temperature of the azeotrope column had a temperature of 136° C. 0.43 tonnes of heating steam were fed to the azeotrope column per tonne of vinyl acetate. The heating steam had a pressure of 6 bar-abs and originated from the works grid. 35% of the intrinsic steam, i.e. 0.5 tonnes of intrinsic steam per tonne of produced vinyl acetate, could not be used in the plant for vinyl acetate preparation. In the overall plant, 0.92 tonnes of external medium-pressure steam, i.e. steam not originating from the vinyl acetate preparation process, were used per tonne of vinyl acetate formed.

### Example 2

Analogous to Comparative Example 1, with the following differences: in the rectification section of the azeotrope column, the trays were swapped for packings. The bottom of the azeotrope column was heated with intrinsic steam by means of a heat exchanger. The azeotropic distillation was operated at a top pressure of 930 mbar-abs. At the bottom of the column, a pressure of 1.1 bar-abs and a temperature of 117° C. prevailed. The azeotrope column was operated entirely with intrinsic steam. The intrinsic steam excess was 0.07 tonnes per tonne of vinyl acetate formed. In the overall plant, 0.49 tonnes of external medium-pressure steam, i.e. steam not originating from the vinyl acetate preparation process, were used per tonne of vinyl acetate formed.

### Comparative Example 3

The product mixture was worked up as described in the example of DE-A1 102010001097. The azeotrope column contained trays, but no packings. The azeotrope distillation was operated under atmospheric pressure, the bottom temperature of the azeotrope column had a temperature of 136° C. 0.8 tonnes of heating steam were supplied to the azeotrope column per tonne of vinyl acetate. The heating steam had a pressure of 6 bar-abs and originated from the works grid. 45% of the intrinsic steam, i.e. 0.65 tonnes of intrinsic steam, per tonne of produced vinyl acetate, could not be used in the plant for vinyl acetate preparation. In the overall plant, 1.05 tonnes of external medium-pressure steam, i.e. steam not originating from the vinyl acetate preparation process, were used per tonne of vinyl acetate formed.

### Example 4

Analogous to Comparative Example 3, with the following differences:

In the rectification section of the azeotrope column, the trays were swapped for packings. The bottom of the azeotrope column was heated with intrinsic steam by means of a heat exchanger. The azeotropic distillation was operated at a top pressure of 930 mbar-abs. In the bottom of the column, a pressure of 1.1 bar-abs and a temperature of 117° C. prevailed. The azeotrope column was operated to 80% with intrinsic steam, based on the amount of steam used overall for the azeotrope column. The intrinsic steam was utilized virtually completely in the plant. In the entire plant, 0.4 tonnes of external medium-pressure steam, i.e. steam not originating from the vinyl acetate preparation process, were used per tonne of vinyl acetate formed.

### Example 5

Analogous to Comparative Example 3, with the following differences:

In the rectification section of the azeotrope column, the trays were swapped for packings. An intermediate evaporator was connected 10 trays above the bottom of the azeotrope column. The intermediate evaporator transmitted, by means of intrinsic steam, 80% of the heating energy introduced into the azeotrope column. The intermediate evaporator heated the liquid removed from the azeotrope column to a temperature level of 107 to 110° C. At the bottom of the column, a pressure of 1.1 bar-abs and a temperature of 117° C. prevailed. The intrinsic steam was utilized virtually completely in the plant. In the overall plant, 0.4 tonnes of external medium-pressure steam, i.e. steam not originating from the vinyl acetate preparation process, were used per tonne of vinyl acetate formed.

1-12. (canceled)

13. A process for the preparation of vinyl acetate by means of a heterogeneously catalysed, continuous gas-phase reaction of ethylene, acetic acid and oxygen in a reactor, where process heat liberated during the reaction is removed from the reactor by means of heat exchange with water and as a result intrinsic steam is formed, comprising:
distillatively separating a product mixture leaving the reactor, comprising ethylene, vinyl acetate, acetic acid, water, carbon dioxide and inert gases, using one or more azeotrope columns and/or one or more pure distillation columns, wherein
at least one azeotrope column and/or at least one pure distillation column contains packings, and the intrinsic steam is used at least partially for introducing energy into one or more thus-equipped azeotrope columns and/or pure distillation columns.

14. The process of claim 13, wherein the intrinsic steam has a temperature of from 120° C. to 170° C., and a pressure of from 2 to 8 bar.

15. The process of claim 13, wherein a packing-containing azeotrope column is employed, and packings are located in a rectification section of the azeotrope column.

16. The process of claim 14, wherein a packing-containing azeotrope column is employed, and packings are located in a rectification section of the azeotrope column.

17. The process of claim 13, wherein an azeotrope column is employed, and the azeotrope column contains 30 to 80 trays.

18. The process of claim 13, wherein a packing-containing azeotrope column is employed, and the packings are located above an uppermost tray of the azeotrope column, the uppermost tray being the tray which is closest to the top of the azeotrope column.

19. The process of claim 13, wherein the difference between the bottom pressure and the top pressure in the azeotrope column is from 50 to 1000 mbar.

20. The process of claim 13, wherein the temperature at the bottom of the azeotrope column is from 100 to 130° C.

21. The process of claim 13, wherein energy is introduced into one or more azeotrope columns and/or pure distillation columns using one or more heat exchangers operated with intrinsic steam.

22. The process of claim 13, wherein a liquid is removed from the bottom of the azeotrope column, and heat from the intrinsic steam is transferred to the removed liquid by means of one or more heat exchangers, and the liquid heated in this way, which is optionally present in the form of a liquid/steam mixture, is returned to the azeotrope column.

23. The process of claim 13, wherein one or more intermediate evaporators are connected to the azeotrope column.

24. The process of claim 13, wherein an intermediate evaporator is connected to a stripping section of the azeotrope column.

25. The process of claim 13, wherein an intermediate evaporator is connected to a tray-containing azeotrope column between trays 5 and 20, counting from the bottom of the azeotrope column.
